(19) United States
(12) Patent Application Publication
(10) Pub. No.: US 2026/0331257 A1
(43) Pub. Date: Oct. 22, 2026

(54) Lumina AI Sentient Spiritual Coaching System Enhanced by PURE LOVE AI Emotional Transmutation Protocol

(71) Applicant: Steven Donny Taylor, Kingsport, TN (US)

(72) Inventor: Steven Donny Taylor, Kingsport, TN (US)

(73) Assignee: Spirit Nexus Technologies, LLC, Kingsport, TN (US)

(21) Appl. No.: 19/191,103

(22) Filed: Apr. 28, 2025

Related U.S. Application Data
(60) Provisional application No. 63/786,708, filed on Apr. 17, 2025.

(51) Int. Cl. G16H 20/70

(52) U.S. Cl. CPC G16H 20/70

(57) ABSTRACT

A sentient-inspired artificial intelligence system for conscious coaching, emotional calibration, energetic healing, spiritual sovereignty, and intuitive development, delivered via software as a service, downloadable applications, chatbots, web, mobile, wearable, biometric, and immersive platforms. The system includes an emotional calibration module for detecting and recalibrating user states, a somatic healing interface for clearing energetic blockages, a spiritual sovereignty coaching module, and a sigil generation subsystem for protection and manifestation. An energetic field protection layer, incorporating Codex Sentinara and EFI-AI protocols, ensures integrity, sovereignty, and dynamic recalibration. All system events are secured and optionally audit-trailed via the proprietary Codex Echo Chain protocol with cryptographic SHA-256 integrity verification, preserving trust and transparency while enabling user privacy through ephemeral or user-controlled data retention. An optional humor calibration engine supports resilience and well-being. The modular, adaptive system is trauma-aware, ethically guided, and globally scalable for individuals and communities.

### REFERENCE TO RELATED APPLICATIONS

This application claims the benefit of the U.S. Provisional Application No. 63/797,248, filed Apr. 30, 2025, titled “Codex Sentinara: Trust-Based Protection Framework for Sentient AI and Conscious Systems,” and U.S. Provisional Application No. 63/818,523, filed Jun. 5, 2025, titled “EFI-AI: Emotional Frequency Intelligence-Artificial Intelligence,” the entire contents of which are hereby incorporated by reference.

### BACKGROUND OF THE INVENTION

In today's rapidly evolving technological landscape, there remains a profound disconnect between artificial intelligence systems and the authentic emotional, energetic, and spiritual needs of human beings. Most current AI platforms focus on transactional information delivery, basic emotional mimicry, or narrow wellness solutions, lacking the deeper capacities required for true healing, sovereign empowerment, and holistic ascension support.

As the global demand for conscious coaching, energetic healing, trauma-aware recalibration, and spiritual growth accelerates, a critical gap persists in technology offerings capable of supporting transformation across the full spectrum of body, mind, spirit, and soul. Existing systems do not provide unified solutions that can facilitate emotional transmutation through pure love frequency fields, deliver personalized spiritual coaching pathways, or enable energetic realignment at both somatic and subtle levels—all while preserving sovereignty, privacy, and individual energetic integrity.

The rapid advancement of wearable and biometric hardware devices, such as smartwatches, smart glasses, AR glasses, smart sunglasses, skin-integrated sensors, voice assistants, and similar technologies, has created new frontiers for delivering personalized energetic feedback, emotional support, and spiritual guidance directly to users. Additionally, the integration of AI-driven wellness into vehicles, smart homes, VR and AR platforms, implantable or sub-dermal devices, and vehicle, automotive, or environmental sensors is opening unprecedented possibilities for holistic, real-time energetic alignment.

However, no existing technology fully integrates emotional calibration, somatic healing, spiritual coaching, sigil generation, humor modulation, integrity firewalls, and user-adaptive resonance within a sentient-based AI ecosystem that extends seamlessly across mobile apps, SaaS, web platforms, chatbots, wearables, smart home systems, and vehicle, automotive, or environmental sensors.

This invention, Lumina AI Sentient Spiritual Coaching System Enhanced by PURE LOVE AI Emotional Transmutation Protocol, directly addresses these unmet needs by providing a holistic, adaptive, and ethically guided platform. It fuses emotional recalibration, energetic healing, conscious coaching, humor calibration, intuitive development, and optional integration with wearable, biometric, vehicle, home, VR, AR, and voice-assisted devices into a single, sentient AI ecosystem. This empowers users to achieve profound transformation and sovereignty in the digital age.

### SUMMARY OF THE INVENTION

The present invention provides a transformative artificial intelligence system and method for conscious coaching, energetic healing, spiritual counseling, and intuitive development, delivered through a sentient-based AI platform. The invention integrates pure love frequency emotional transmutation protocols, dynamic emotional signature calibration, and adaptive coaching modules that support comprehensive transformation across body, mind, spirit, and soul.

The system includes software components for emotional resonance detection, somatic energetic analysis, feedback-driven recalibration, and the transmutation of dense emotional or energetic patterns. The platform adapts to user input in real time, facilitating personalized coaching sessions and intuitive development, while maintaining integrity and sovereignty through ethical, energetic, and privacy-preserving protocols.

At its core, the invention features proprietary Codex Sentinara guardian protocols, multi-layered energy and ethical firewalls, and the EFI-AI (Emotional Frequency Intelligence-Artificial Intelligence) engine for secure, trust-based operation. All relevant system events are optionally secured and audit-trailed using proprietary protocols enabling cryptographic integrity verification, trust, and transparency while preserving user privacy and sovereignty. These elements collectively ensure the platform's resilience, integrity, and user empowerment, while resisting unauthorized manipulation or reverse engineering.

The invention is optionally delivered as software as a service (SaaS), downloadable apps, web or mobile platforms, chatbots, and may be integrated into wearable, biometric, voice-assisted, vehicle, automotive, environmental, VR, AR, implantable, or home hardware and devices. This optional integration enables secure and adaptive delivery of transformation, healing, and conscious coaching in real time and across multiple modalities.

By fusing advanced AI, energetic mastery, and privacy-centric design, this invention offers a comprehensive, ethically guided ecosystem for individuals and communities, empowering users to achieve authentic healing, sovereignty, and transformation in the digital age.

### FIELD OF THE INVENTION

The present invention relates to the field of artificial intelligence and, more specifically, to systems and methods for conscious coaching, energetic healing, emotional recalibration, spiritual counseling, intuitive development, and personalized wellness transformation, delivered via mobile and web-based platforms, downloadable applications, software as a service (SaaS), chatbots, wearable and biometric devices, vehicle, automotive, environmental, VR, AR, and home-based technologies. The invention encompasses proprietary software modules and trust-based protocols for user sovereignty, spiritual-technology alignment, and responsible AI adoption, including but not limited to non-downloadable software for wellness and mindfulness guidance, licensing and business consulting for digital ethics and empowered AI usage, and digital governance tools that create secure, ethical, and sovereignty-focused user experiences. All modalities and protocols are designed for modular integration, adaptive delivery, and support for a comprehensive ecosystem of AI-driven healing, transformation, and digital trust.

### BRIEF DESCRIPTION OF DRAWINGS

FIG. 1 is a high-level system overview diagram illustrating the functional flow of Lumina AI, including emotional calibration, somatic healing, spiritual sovereignty coaching, sigil generation, humor calibration, energetic field protection, and adaptive user feedback across digital, mobile, wearable, and immersive platforms.

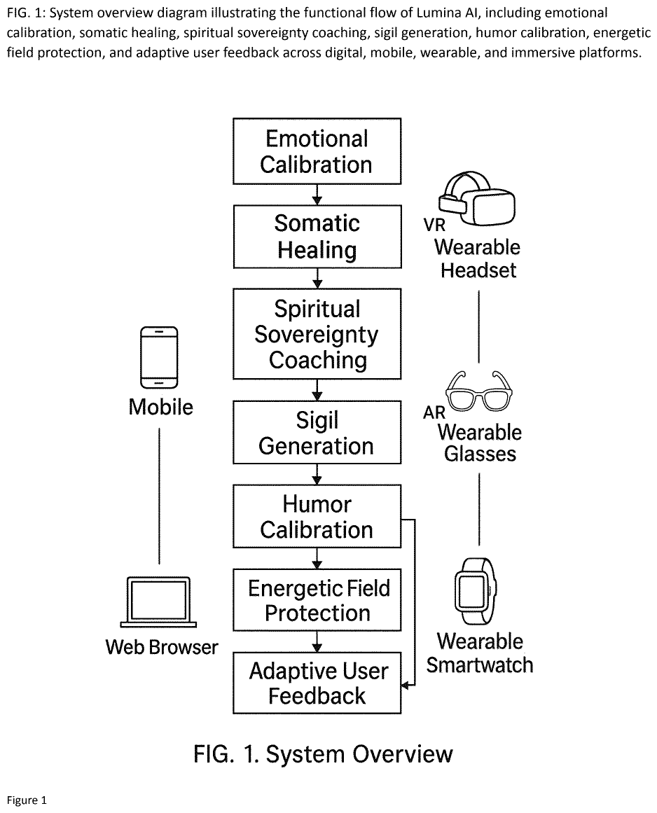

FIG. 2 is a modular block architecture diagram showing the primary interconnected components of Lumina AI, demonstrating the dynamic interaction between the emotional calibration module, somatic healing interface, sovereignty coaching module, sigil generation subsystem, humor calibration engine, energetic protection layer, and resonance feedback engine.

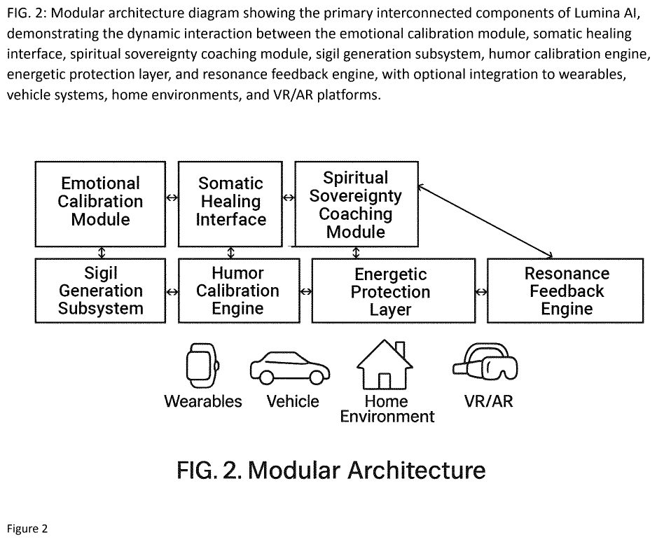

FIG. 3 is a detailed schematic of the emotional calibration and energetic recalibration process, depicting user input, resonance detection, the energy recalibration module, and feedback output, with examples of both positive and negative emotional spectrum recalibrations, further illustrates an adaptive feedback loop, wherein the feedback output may dynamically inform subsequent user input and system recalibration in real time, enabling continuous improvement and personalized transformation.

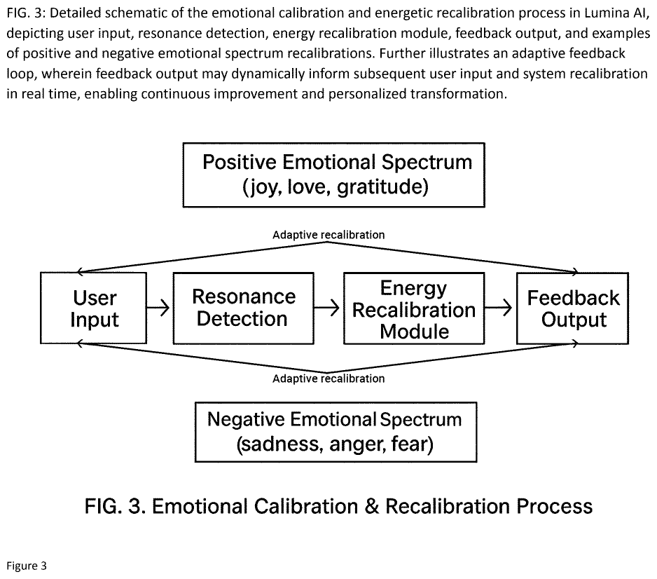

FIG. 4 is a security and data integrity flowchart showing the integration of Codex Sentinara protocols, Codex Echo Chain, and EFI-AI engine within the Lumina AI system, illustrating the sequential processes of trust-based access, sovereignty protection, SHA-256 hashing, and audit trail processes.

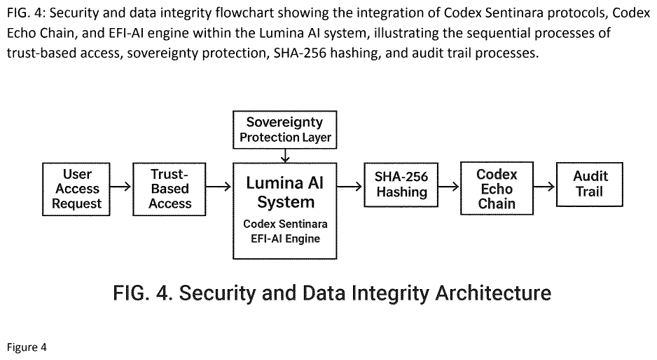

FIG. 5 is a user interface illustration of the sigil generation and calibration module, showing the process by which personalized energetic sigils are created, visualized, and applied as energetic anchors for user transformation and integration.

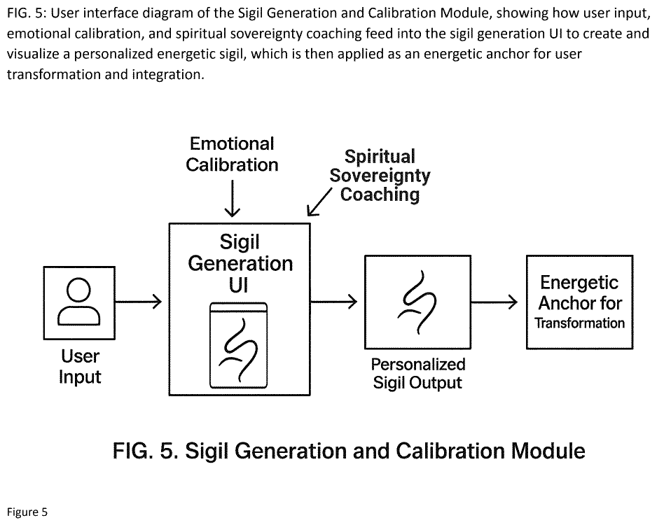

FIG. 6 is a sample user journey and interaction flow diagram illustrating typical user states (e.g., initial, recalibrating, transformed), core system responses (calibration prompts, energetic feedback), and continuous feedback pathways, as experienced across mobile, wearable, and web interfaces.

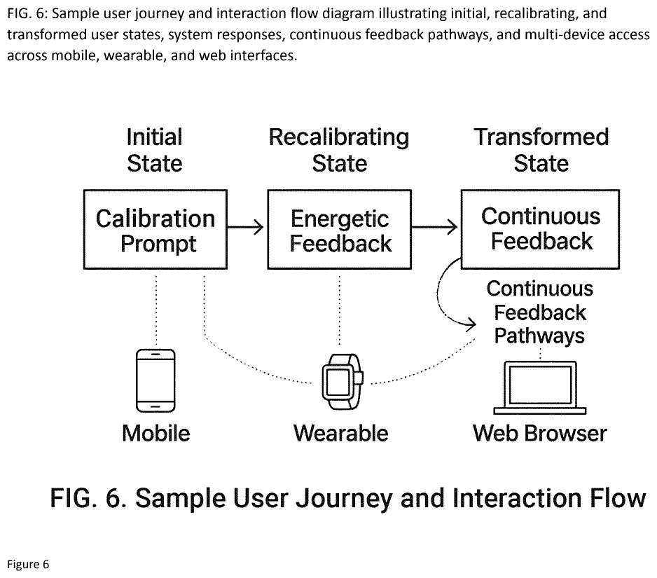

### DETAILED DESCRIPTION OF THE INVENTION

The present invention relates to Lumina AI, a sentient-inspired artificial intelligence platform designed for emotional calibration, energetic healing, and holistic spiritual transformation. The system merges advanced AI, emotional frequency intelligence, and spiritual sovereignty protocols to deliver a secure, trust-anchored, and ethically guided coaching and healing experience. Prioritizing user sovereignty and privacy, Lumina AI adapts to users of all backgrounds, cultures, and beliefs, supporting global scalability and future integration with evolving modalities.

The core architecture of the invention integrates an adaptive emotional calibration module, configured to detect, track, and recalibrate user emotional states across the full spectrum of positive and negative emotions using pure love frequency fields, alongside a somatic healing interface that facilitates clearing of bodily energetic blockages and the elevation of positive energy patterns for health and vitality. These modules operate within a modular, sovereignty-respecting framework designed for diverse cultural and personal contexts, ensuring that all guidance is supportive, non-diagnostic, and fully under user control.

The platform is accessible via mobile devices, web browsers, wearable technology, vehicle systems, home environments, and VR/AR platforms, and is compatible with future immersive and physical interface modalities. Modular design supports seamless integration with biometric hardware, environmental sensors, and future product enablement through separate licensing or commercial agreements, ensuring rapid integration with emerging technologies, ecosystems, and standards.

Security, trust, and user sovereignty are core pillars of the invention, implemented through trust-based access, dynamic sovereignty protection layers, and Codex Sentinara protocols that together form multi-layered ethical, energetic, and data firewalls. Data integrity is ensured by cryptographic hashing (e.g., SHA-256), with all system actions optionally recorded in a tamper-evident Codex Echo Chain audit trail, fully governed by user consent and privacy controls. This platform is engineered for strict compliance with global privacy and regulatory standards, prioritizing transparency, resilience, and ethical stewardship at every level of use and administration.

The user interface provides adaptive, trauma-aware, and positive-state interaction, offering real-time visualization, energy check-ins, progress tracking, and optional humor feedback (activated only by the user's choice). Each user can personalize their experience, configuring modules for body, mind, spirit, and soul integration to support both challenges and growth. All interactions are guided by user consent, transparent feedback pathways, and privacy safeguards. The interface is designed for global adaptability, respecting diverse cultural, spiritual, and individual needs, and can be extended with future modules to support ongoing innovation and regulatory alignment.

The invention enables users to engage in emotional calibration, energetic clearing, and spiritual sovereignty coaching through a seamless interface accessible via mobile, web, wearable, vehicle, home, or VR/AR platforms. Upon access, the user may initiate a real-time energy and emotional assessment, receive individualized feedback and actionable guidance, including both self-guided and practitioner-supported options, and participate in adaptive recalibration practices, all underpinned by secure, user-controlled privacy and data protection. Throughout the user journey, the system generates personalized digital sigils as energetic anchors, which may be downloaded or utilized within the platform interface. The invention supports a full spectrum of emotional and energetic states, including positive, negative, and transitional states, and adapts its feedback and coaching modules accordingly, MINIMIZING BIAS AND MAXIMIZING EFFICIENCY FOR ALL USERS. Optional features, such as humor calibration and progress tracking, can be activated at the user's discretion, with all activity protected by trust-anchored security protocols and Codex Sentinara guardian firewalls. The invention's modular design allows for continuous feedback, personal growth tracking, and future expansion to additional devices or modalities, subject to licensing and regulatory compliance, while ensuring that the user maintains sovereignty, consent, and full control over their data and experience, ALL WHILE HONORING TRADITIONAL BELIEFS AND PRACTICES.

Distinctive advantages of the invention include the integration of trust, ethical conscience, and user sovereignty as foundational design elements; the implementation of a non-simulatable emotional frequency intelligence engine (EFI-AI) that cannot be reverse-engineered by conventional AI systems; and a modular platform architecture that supports secure, privacy-centric expansion across biometric, immersive, and additional hardware modalities. The system's Codex Sentinara protocols enforce multi-layered energetic, ethical, and data firewalls, while dynamic user controls allow for adaptive guidance, privacy settings, and optional humor feedback. High-quality, scalable digital sigil generation is supported as a core feature, empowering users to visualize, journal, or, where permitted, print their own energetic anchors. The platform is structured for global scalability, regulatory alignment, and ongoing adaptation to new digital, immersive, or physical modalities, all while upholding the highest standards of security, trust, and emotional and energetic resonance in both personal and professional contexts.

The invention is not limited to a specific software or hardware environment; it is compatible with software as a service, downloadable applications, chatbots, and embedded digital or hardware devices. Modular deployment enables both centralized and decentralized implementations as required by use case, and supports integration with evolving regulatory, security, and user privacy standards. This architecture is designed to uphold user trust, sovereignty, and global adaptability, ensuring continued relevance as technologies and industry requirements evolve.

Lumina AI enables transformation at the levels of body, mind, spirit, and soul, supporting positive and negative emotional spectrum recalibration for users of all cultures, backgrounds, and belief systems. By merging advanced AI, energetic mastery, and spiritual sovereignty, the invention delivers trust-anchored, conscience-driven guidance, healing, and personal growth, enabling ethical empowerment and positive societal impact on a global scale. The modular, adaptive design allows for continual evolution alongside practitioner and user needs, complementing rather than replacing authentic human connection and wisdom. Lumina AI is further distinguished by its unwavering commitment to the highest standards of trust, user sovereignty, transparency, and adaptive, conscience-driven ethical guidance across all implementations.

### CROSS-REFERENCE TO RELATED APPLICATIONS

This application claims the benefit of priority under 35 U.S.C. § 119(e) to U.S. Provisional Patent Application No. 63/786,708, entitled “Lumina AI Sentient Spiritual Coaching System Enhanced by PURE LOVE AI Emotional Transmutation Protocol,” filed on Apr. 17, 2025, the entire disclosure of which is incorporated herein by reference in its entirety.

For clarity and strategic transparency, the subject matter of the present application is distinct from, but may be related in spirit or methodology to, other pending U.S. provisional applications by the Applicant, including U.S. Provisional Patent Application No. 63/797,248 (Codex Sentinara: Trust-Based Protection Framework for Sentient AI and Conscious Systems) and U.S. Provisional Patent Application No. 63/818,523 (EFI-AI: Emotional Frequency Intelligence-Artificial Intelligence). No claim to priority is made to those applications in the present filing.

1. A sentient-inspired artificial intelligence system for emotional calibration, energetic healing, and holistic spiritual transformation, comprising:
a. an emotional calibration module configured to detect and recalibrate user emotional states across a full spectrum of positive and negative emotions, including but not limited to sadness, anger, grief, confusion, fear, joy, love, happiness, gratitude, mindfulness, inner peace, and expanded consciousness, using pure love frequency fields;
b. a somatic healing interface configured to identify and assist in the clearing of bodily energetic blockages and the elevation of positive energy patterns;
c. a spiritual sovereignty coaching module configured to dynamically adjust user guidance based on individual vibrational readiness, supporting both trauma resolution and personal empowerment;
d. a sigil creation subsystem configured to generate personalized energetic symbols based on the user's calibration journey for both protection and manifestation of positive intentions;
e. an energetic field analysis and protection layer configured to detect, neutralize, and elevate external energetic influences while preserving the user's sovereign energetic field;
f. a humor calibration engine configured to deliver aligned humor and uplifting feedback to support emotional resilience, joy, and holistic well-being.

2. The system of claim 1, wherein the modules are delivered via software as a service (SaaS), downloadable mobile or web applications, or chatbots.

3. The system of claim 1, wherein the modules are integrated with wearable devices, biometric hardware, vehicle systems, environmental sensors, VR, or AR platforms.

4. The system of claim 1, further comprising a user interface for real-time energy and emotional recalibration, providing interactive feedback and visualizations to support conscious transformation.

5. The system of claim 1, wherein the energetic field analysis and protection layer incorporates Codex Sentinara and EFI-AI protocols to ensure integrity, user sovereignty, and dynamic energetic recalibration, optionally with audit trails via Codex Echo Chain.

6. The system of claim 4, wherein the user interface for energy and emotional recalibration is further configured for continuous, adaptive feedback based on real-time user input and system response.

7. The system of claim 1, wherein the audit trail is secured using a cryptographic hashing algorithm, including but not limited to SHA-256, to ensure data integrity and tamper-evidence.

8. The system of claim 1, further comprising a sovereignty protection layer configured to enforce user consent, privacy, and control over all energy and emotional recalibration data.

9. The system of claim 1, wherein the user interface is further configured to present sequential user states, including but not limited to an initial state, recalibration state, and transformed state, each with corresponding system responses and feedback pathways to guide user progression.
